(19) United States
(12) Patent Application Publication
(10) Pub. No.: US 2007/0016128 A1
(43) Pub. Date: Jan. 18, 2007

(54) Two-component dispensing system comprising a dispenser and a mixer

(75) Inventor: Wilhelm A. Keller, Merlischachen (CH)

Correspondence Address: FOLEY AND LARDNER LLP;SUITE 500, 3000 K STREET NW, WASHINGTON, DC, 20007, US

(73) Assignee: Mixpac Systems AG

(21) Appl. No.: 10/556,074

(22) PCT Filed: May 14, 2004
(86) PCT No.: PCT/CH04/00295

§ 371 Date: Nov. 9, 2005

(30) Foreign Application Priority Data
May 19, 2003 (CH) 2003 0884/03

(51) Int. Cl. A61M 37/00

(52) U.S. Cl. 604/89; 604/213; 366/130

(57) ABSTRACT

The dispensing assembly for two components comprises a dispensing cartridge or a dispensing appliance and a mixer, a transfer portion (3) being interposed between the dispensing cartridge (1) or appliance and the mixer (5) which is connectable both to the cartridge or appliance and to the mixer. In a preferred embodiment, respective adapters (10, 21) are provided on either side of the transfer portion. The transfer portion (3) comprises a cylindrical bar (38) at the periphery of which two longitudinal grooves (39, 40) are arranged and which is enclosed in a jacket (28) in order to form two channels (17, 18) that are tight such that no material may pass from one channel to the other. An assembly of this kind allows a simple manufacture of the transfer portion and a reliable operation, which is important particularly in laparoscopy as the distance between the dispensing cartridge and the operating field in the abdominal cavity is quite large in this case.

In most dispensing assemblies, the mixer is fastened to the cartridge and the assembly is used in this form. Often an accessory is attached to the mixer to influence the shape of the mixed components and to allow a targeted application of the material to the intended location in special situations. However, there are a number of applications, more particularly in medicine, where this simple assembly of dispensing cartridge, mixer, and accessory device is insufficient.

In medicine, certain joinings are now being sealed rather than sutured, for which purpose a two-component sealant is used. Thus, for example, the application of dispensing assemblies in operations involving the use of an endoscope requires a long transfer portion between the cartridge and the mixer, particularly in the case of laparoscopy. In laparoscopy, relatively long tubes are introduced into the abdominal cavity, so that quite a long distance between the cartridge and the dispensing tip may result.

WO-A2-03/039375 discloses a spraying device for laparoscopy according to the preamble of claim 1, comprising a tubular transfer portion adapted to be coupled with a dispensing device on its inlet side and with a spraying device on its outlet side, the spraying device including a mixing chamber with a flexible mixing member. The tubular transfer portion comprises two longitudinal bores. The manufacture of tubes having thin longitudinal bores may become problematic if longer tubes are required.

WO-A1-99/32173 discloses an applicator for spray dispensing biological liquids, comprising an elongated body adapted to be connected to a dispensing device on its inlet side and to a spraying head on its outlet side. This applicator is neither intended nor suitable for laparoscopy.

WO-A-01/74253 discloses a dispensing device comprising a dispensing tip having two rigid portions that are connected to each other by a flexible section. This device is not intended for laparoscopy.

On the background of this prior art, it is the object of the present invention to provide a dispensing assembly where a dispensing cartridge or a dispensing appliance can be connected by means of a transfer portion to a relatively remote mixer or another accessory, e.g. a spraying head, and that can be manufactured inexpensively. This is accomplished by the assembly as described in claim 1.

The invention will be explained in more detail hereinafter with reference to drawings of an exemplary embodiment.

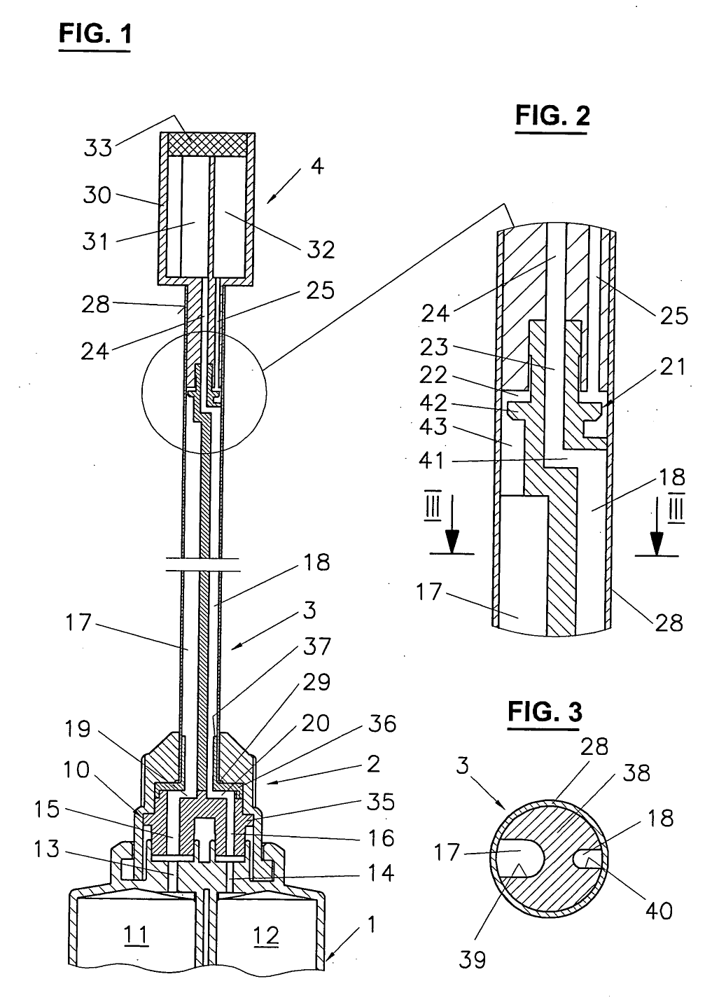

FIG. 1 shows an assembly of the invention with an attached compensating device in a longitudinal section;

FIG. 2 shows an enlarged detail of FIG. 1;

FIG. 3 shows a section according to line III-III in FIG. 2;

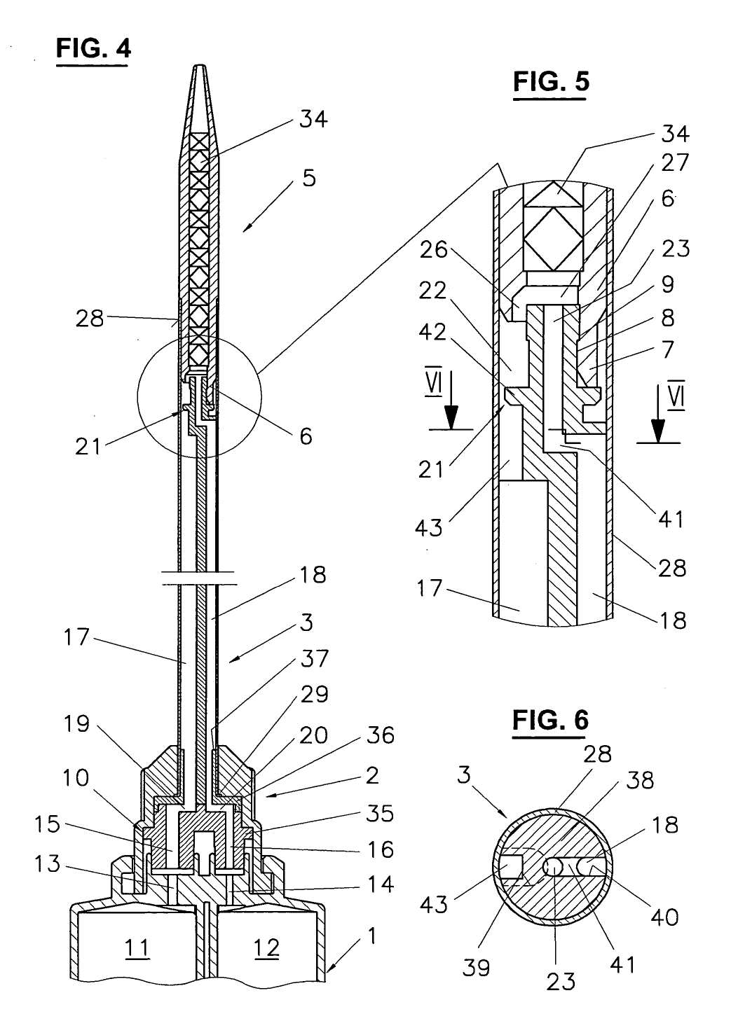

FIG. 4 shows a dispensing assembly of the invention with the mixer attached;

FIG. 5 shows an enlarged detail of FIG. 3; and

FIG. 6 shows a section according to line VI-VI in FIG. 5.

FIG. 1 shows a double cartridge 1 with a coupling ring 2 for the attachment of a transfer portion 3 and a compensating device 4 fitted to the latter. The coupling ring and the cartridge comprise bayonet securing means that are known per se. Other securing means such as e.g. snap rings are also possible. The compensating device serves to mutually adjusting the component levels to ensure an accurate mixing ratio of the components from the beginning of the dispensing or mixing operation, respectively, and for a complete air evacuation of the double cartridge to prevent any air inclusion therein. These two conditions are particularly important in the field of medicinal technique.

For an application in endoscopy respectively laparoscopy, the transfer portion and the mixer may not exceed a certain small diameter and must be free of stepped portions. However, for economic reasons, commercially available cartridges are used which are also intended for other applications and whose adjacent or concentric outlets come to a total diameter that is substantially larger than the allowable diameter of the transfer portion, and therefore an adapter is required between the cartridge and the transfer portion to allow the passage of the material between the cartridge outlets and the channels of the transfer portion. Moreover, it is advisable also to provide an adapter between the transfer portion and the mixer or the compensating device.

Each storage cylinder 11 and 12 of the double cartridge comprises an outlet 13 and 14 leading to respective channels 15 and 16 in entrance adapter 10. In the exemplary embodiment, the volumes resp. the cross-sectional areas and the outlets of the storage cylinders are different from each other, the ratio varying between 1:1 and 1:10 according to the intended application and the composition.

For reasons related to the production technique, entrance adapter 10 is made of two parts and composed of adapter body 35 with channels 15 and 16 and on the outlet side of a flange portion 36 on the tubular portion 37 of which the jacket 28 of transfer portion 3 is fitted.

The transfer portion consists of a cylindrical bar 38 surrounded by a jacket 28. Bar 38 is provided with two opposed grooves 39 and 40 forming two channels 17 and 18 together with the jacket, see FIG. 3. Care must be taken here that the two grooves or channels are tightly separated from each other to prevent that material may pass from one channel to the other. Tightness may e.g. be achieved by press fitting the jacket onto the bar, but other sealing methods such as a sealing bead or cementing are also possible.

In the present example, channels 17 and 18 are at a smaller mutual distance than the two outlets 13 and 14 of the cartridge, and channels 15 and 16 of the entrance adapter are shaped such that its outlets 19 and 20 match channels 17 and 18 of the transfer portion. On the cartridge side, the end of jacket 28 has a flanging 29 for being retained by the shoulder of the coupling ring, as appears in FIG. 1 or 4.

At the other end of the transfer portion, an exit adapter 21 is located with channels 22 that are arranged concentrically with respect to central channel 23 and whose outlets communicate either with inlets 24 and 25 of the compensating device or with inlets 26 and 27 of the mixer. Jacket 28 extends beyond the exit adapter, thereby allowing either the correspondingly shaped inlet of the compensating device or the mixer inlet to be inserted in this tube section while additionally providing a stabilization of the mixer.

As appears in FIG. 6, external channel 18 is connected to approximately centrally arranged channel 23 by a transversal channel 41. Channel 17 located at the left of the drawing ends in a rectangular channel 43 extending up to a surrounding shoulder 42 in the exit adapter, the shoulder having a smaller diameter than the jacket, thereby allowing the material to flow around it. On top of surrounding shoulder 42, channels 22 extend concentrically around channel 23. Furthermore, shoulder 42 serves as a rest for the mixer.

Compensating device 4 comprises an enclosure 30 as well as two chambers 31 and 32, and it is terminated by a porous filter 33 that is essentially air permeable only but capable of retaining the material. It is also possible to provide a cap having one or a plurality of capillary opening(s) instead of a porous filter. The enclosure of the compensating device is preferably made of a transparent material, thereby allowing to visually ascertain whether the levels of the two storage cylinders are aligned and the transfer portion is completely filled with the components. It is important here that the components arrive in the mixer simultaneously so that a sealant mixture is achieved from the beginning of the dispensing operation in the abdominal cavity.

In FIGS. 1 and 2, the compensating device is attached to the transfer portion und it appears that channels 22 and 23 of the exit adapter communicate with inlets 24, 25 of the compensating device. After having pulled off the compensating device, the filling levels of the two components are at different heights, thereby ensuring that no reaction may take place at this time.

In FIGS. 4 and 5, the same cartridge and the same coupling ring as well as the same transfer portion with exit adapter 21 are shown whereas a mixer 5 is attached to the transfer portion. The inlet of the mixer comprises a connecting portion 6 having elastic tongues 7 with shoulders 8 which engage behind a corresponding stepped portion 9 in the exit adapter and prevent that the mixer may be pulled out after having been inserted in the tube of the transfer portion. The mixer comprises the known mixer elements 34.

As appears in FIGS. 4 and 5, centrally arranged channel 23 of the exit adapter is concentrically surrounded by other channels 22 resp. by the tongues of the mixer inlet, the channels communicating with mixer inlets 26 and 27.

The sequence of operations may be described as follows: After having removed the closure cap from the filled cartridge, the preassembled transfer portion with the entrance and exit adapters is secured to the cartridge by means of the coupling ring. Then the compensating device is introduced into the transfer portion as far as possible and the entire assembly is directed vertically upwards with the compensating device at the top to allow air evacuation.

Subsequently, material is dispensed until both components are visible in the compensating device. It is thereby ensured that both components reach the mixer from the very beginning of the dispensing operation so that a sealant mixture is obtained from the beginning. Furthermore, it is achieved by these measures that also the two storage cylinders of the cartridge and the transfer portion are free of air.

Then the compensating device is removed and disposed of, and the connecting portion of the mixer is introduced into the tube of the transfer portion and pushed in until it snaps in, whereby the mixer is operative.

Due to the different design of the channels in the outlet portion and of those in the inlet portion of the compensating device or in the attached mixer, respectively, it is avoided that the two components may come into contact with each other.

In the preceding exemplary embodiment, a cartridge having side-by-side outlets and side-by-side inlets of the entrance adapter, as well as side-by-side channels of the transfer portion have been described which end in concentric channels of the exit adapter and lead to the concentric inlets of the mixer or of the compensating device. However, it is also possible to apply this system of cartridge, transfer portion, and compensating device or mixer in an arrangement where the two components are always or partly disposed concentrically while the adapters are correspondingly designed.

Furthermore it is possible to extend the dispensing assembly to the application of three components.

With regard to the application of the assembly of the invention in endoscopy it is understood that the dimensions and the configuration of the transfer portion and of the mixer must be adapted to the internal diameter of the endoscopic tubes while no part of the mixer may have a greater external diameter than that of the tube of the transfer portion but may be conically tapered toward the outlet. In other applications it is of course possible to choose different dimensions.

1. A dispensing assembly for two components, including a dispensing cartridge or a dispensing appliance and a mixer, a transfer portion being interposed between the dispensing cartridge or appliance and the mixer which is connectable both to the cartridge or appliance and to the mixer and comprises at least two ducts, wherein the transfer portion comprises a cylindrical bar at the periphery of which two longitudinal grooves are arranged, and in that the bar is enclosed in a jacket in order to form two channels that are tight such that no material may pass from one channel to the other.

2. A dispensing assembly according to claim 1, wherein a respective adapter is provided on each side of the transfer portion.

3. A dispensing assembly according to claim 1, wherein the adapter comprises inlets at one end and outlets at the other end, which communicate with the outlets of the cartridge resp. with the inlets of the compensating device or of the mixer, on one hand, and with the channels of the transfer portion.

4. A dispensing assembly according to claim 1, wherein the mixer inlet is provided with a mixer connecting portion which is insertable into the projecting tube of the transfer portion and connectable to the exit adapter on the transfer portion, and which comprises elastic tongues that are provided with respective shoulders cooperating with corresponding shoulders in the exit adapter.

5. A dispensing assembly according to claim 4, wherein the exit adapter comprises two concentrically disposed outlets and the mixer inlet comprises two concentrically disposed inlets.

6. A dispensing assembly according to claim 1, wherein it comprises a compensating device that is connectable to the exit adapter provided on the transfer portion.

7. A dispensing assembly according to claim 1, wherein the compensating device comprises an enclosure having two separate chambers that are made of a transparent material, the enclosure being terminated by a porous filter or one or a plurality of capillary opening(s).

8. A dispensing assembly according to claim 1, wherein the cartridge outlets and/or the transfer portion inlets and/or channels and/or outlets are arranged side by side or concentrically.

9. A dispensing assembly according to claim 1, characterized in that the inlets of the mixer and/or the compensating device are arranged side by side.

10. An application of the dispensing assembly according to claim 1 in conjunction with an endoscopic instrument, wherein the diameters of the transfer portion and of the mixer are adapted to the internal diameter of the endoscopic instrument, and in that the external diameter of the mixer is no greater than the external diameter of the tube of the transfer portion.
